SEQUENCE LISTING (13 sequences)

SEQ ID NO: 1 - protein, 363 aa, Ricinus communis
  misc_feature: ricinus communis delta 9 180 Acyl ACP Desaturase
ASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 2 - DNA, 1092 bp, Ricinus communis
  misc_feature: residues 138 to 1239 of open reading frame
gcctctaccctcaagtctggttctaaggaagttgagaatctcaagaagcctttcatgcctcctcgggaggtacatgttcaggttacccattctatgccaccccaaaagattgagatctttaaatccctagacaattgggctgaggagaacattctggttcatctgaagccagttgagaaatgttggcaaccgcaggattttttgccagatcccgcctctgatggatttgatgagcaagtcagggaactcagggagagagcaaaggagattcctgatgattattttgttgttttggttggagacatgataacggaagaagcccttcccacttatcaaacaatgctgaataccttggatggagttcgggatgaaacaggtgcaagtcctacttcttgggcaatttggacaagggcatggactgcggaagagaatagacatggtgacctcctcaataagtatctctacctatctggacgagtggacatgaggcaaattgagaagacaattcaatatttgattggttcaggaatggatccacggacagaaaacagtccataccttgggttcatctatacatcattccaggaaagggcaaccttcatttctcatgggaacactgcccgacaagccaaagagcatggagacataaagttggctcaaatatgtggtacaattgctgcagatgagaagcgccatgagacagcctacacaaagatagtggaaaaactctttgagattgatcctgatggaactgttttggcttttgctgatatgatgagaaagaaaatttctatgcctgcacacttgatgtatgatggccgagatgataatctttttgaccacttttcagctgttgcgcagcgtcttggagtctacacagcaaaggattatgcagatatattggagttcttggtgggcagatggaaggtggataaactaacgggcctttcagctgagggacaaaaggctcaggactatgtttgtcggttacctccaagaattagaaggctggaagagagagctcaaggaagggcaaaggaagcacccaccatgcctttcagctggattttcgataggcaagtgaagctgtag

SEQ ID NO: 3 - DNA, 34 bp, Artificial Sequence
  misc_feature: PCR primer; sequence flanking unique XbaI site at the 5′ end of the open reading frame
gtgagcggataacaatttcacacagtctagaaat

SEQ ID NO: 4 - DNA, 72 bp, Artificial Sequence
  misc_feature (56)..(57): PCR primer is a degenerate oligonucleotide in which “n” indicates the presence of either C, A, T or G at that nucleotide position
ccaaattgcccaagacgtcggacttgcacctgtttcatcccgaactccatccaamnnattcagcattgtttg

SEQ ID NO: 5 - DNA, 31 bp, Artificial Sequence
  misc_feature: PCR primer
gaaacaggtgcaagtccgacgtcttgggcaa

SEQ ID NO: 6 - DNA, 26 bp, Artificial Sequence
  misc_feature: PCR primer
gttttctgtccgcggatccattcctg

SEQ ID NO: 7 - DNA, 34 bp, Artificial Sequence; identical to SEQ ID NO: 3
  misc_feature: PCR primer

SEQ ID NO: 8 - DNA, 30 bp, Artificial Sequence
  misc_feature: PCR primer
cacgaggccctttcgtcttcaagaattctc

SEQ ID NO: 9 - DNA, 28 bp, Artificial Sequence
  misc_feature: PCR primer
ttgataagtgggaagggcttcttccgtt

SEQ ID NO: 10 - DNA, 66 bp, Artificial Sequence
  misc_feature (41)..(43): PCR primer is a degenerate oligonucleotide in which “n” indicates the presence of either C, A, T or G and in which “k” indicates the presence of either T or G.
aacggaagaagcccttcccacttatcaaacannkctgaatnnknnkgatggagttcgggatgaaac

SEQ ID NO: 11 - DNA, 26 bp, Artificial Sequence
  misc_feature: PCR primer
tccattcctgaaccaatcaaatattg

SEQ ID NO: 12 - DNA, 70 bp, Artificial Sequence
  misc_feature (22)..(24): PCR primer in a degenerate oligonucleotide in which “n” indicates the presence of either C, A, T or G at that nucleotide position and in which “k” indicates the presence of either T or G at that nucleotide position.
ttgattggttcaggaatggatnnkcggnnkgaaaacagtccataccttnnkttcatctatacatcattcc

SEQ ID NO: 13 - DNA, 30 bp, Artificial Sequence
  misc_feature: PCR primer
gcaaaagccaaaacggtaccatcaggatca

(19) United States
(12) Patent Application Publication
(10) Pub. No.: US 2002/0151019 A1
(43) Pub. Date: Oct. 17, 2002

(54) Mutant fatty acid desaturase and methods for directed mutagenesis

(76) Inventor: John Shanklin, Shoreham, NY (US)

Correspondence Address: Margaret C. Bogosian, Patent Counsel, Brookhaven National Laboratory, Bldg. 475D, P. O. Box 5000, Upton, NY, 11973-5000, US

(21) Appl. No.: 10/017,145

(22) Filed: Dec. 14, 2001

Related U.S. Application Data
(63) Continuation-in-part of application No. 09/328,550, filed on Jun. 9, 1999, now abandoned.
Application No. 09/328,550 is a continuation-in-part of application No. 09/233,856, filed on Jan. 19, 1999, now abandoned.

(51) Int. Cl. C12N 9/04; C07H 21/04; C12P 7/64; C12P 21/02; C12N 5/04

(52) U.S. Cl. 435/190; 435/410; 435/320.1; 435/69.1; 536/23.2; 435/134

(57) ABSTRACT

The present invention relates to methods for producing fatty acid desaturase mutants having a substantially increased activity towards substrates with fewer than 18 carbon atom chains relative to an unmutagenized precursor desaturase having an 18 carbon chain length specificity and to the fatty acid desaturases that are produced by the methods. The present invention further relates to a method for altering a function of a protein, including a fatty acid desaturase, through directed mutagenesis involving identifying candidate amino acid residues, producing a library of mutants of the protein by simultaneously randomizing the amino acid at each candidate position, and selecting for mutants which exhibit the desired alteration of function. Candidate residues are identified by a combination of methods including random mutagenesis, structural analysis of the protein, and sequence analysis of the protein. Enzymatic, binding, structural and other functions of proteins can be altered by the method.

### RELATED APPLICATIONS

The present application is a continuation-in-part of U.S. patent application Ser. No. 09/328,550 filed on Jun. 9, 1999, which was a continuation-in-part of U.S. patent application Ser. No. 09/233,856 filed on Jan. 19, 1999.

This invention was made with Government support under contract number DE-AC02-98CH10886, awarded by the U.S. Department of Energy. The Government has certain rights in the invention.

### BACKGROUND OF THE INVENTION

Fatty acid biosynthesis in higher plants has recently attracted increased interest because of the possible use of plant oils as renewable sources for reduced carbon. The diversity of fatty acid forms in wild plants is vast compared to that of crop plants. This diversity is reflected in the variations in chain length, the number and position of double bonds, and the position and occurrence of a variety of other functional groups in the fatty acids of wild plants.

In plants, fatty acid biosynthesis occurs in the chloroplasts of green tissue or in the plastids of non-photosynthetic tissues. The primary products in most plants are acyl carrier protein (ACP) esters of the saturated palmitic (palmitoyl-ACP) and/or stearic (stearoyl-ACP) acids, palmitic acid having a 16 carbon atom chain length and stearic acid having an 18 carbon atom chain length. Two types of desaturase molecules are involved in the production of monounsaturated fatty acids (monoenes), soluble, and integral membrane proteins. Desaturases are specific for a particular substrate carbon atom chain length (chain length specificity) and introduce the double bond between specific carbon atoms in the chain (double bond positional specificity) by counting from the carboxyl end of the fatty acid. For instance, the castor Δ⁹-18:0 desaturase is specific for stearoyl-ACP, and introduces a double bond between carbon atoms 9 and 10.

The introduction of non-native desaturase isoforms having unique characteristic chain length and double bond positional specificities into agricultural crops offers a way to manipulate the content, physical properties and commercial uses of plant-produced oils. Unfortunately, the introduction of non-native acyl-ACP desaturase isoforms into agricultural crop plants has yet to lead to the efficient production of unusual or uniquely useful monoenes by agricultural crop plants. An alternative way in which to accomplish the manipulation of the content, physical properties and commercial uses of oilseed crops would be through the introduction of a native desaturase which had been manipulated in such a way as to alter its chain length and/or double bond positional specificities.

As the genes encoding more desaturase enzymes are identified it is becoming apparent that many of the different activities are derived from relatively few common archetypes encoding the soluble and membrane classes of desaturases.

Molecular modeling and X-ray crystallographic studies of soluble acyl-ACP desaturases have identified amino acid residues within the substrate binding channel which are in very close proximity to the fatty acid substrate. Such residues are referred to as “contact residues”. That earlier research demonstrated that certain modifications of one or more contact residues and modification of some non-contact residues can alter the in vitro chain-length and double bond positional specificities of acyl-ACP desaturases (Cahoon, et al. Proc. Natl. Acad. Sci. USA (1997) 94:4872-4877 and Cahoon, et al. U.S. Pat. Nos. 5,705,391, 5,888,790 and 6,100,091). Those studies were carried out using predictions formulated from the three dimensional structure of the castor Δ⁹-18:0 acyl-ACP desaturase in combination with alignment of its sequence with that of a Δ⁶-16:0 acyl-ACP desaturase as well as with the sequences of other 18:0 desaturases. The studies examined the effects of replacing specific contact and non-contact amino acid residues of the Δ⁶-16:0 desaturase with various amino acid residues in cognate positions in the Δ⁹-18:0 desaturase on the in vitro substrate chain length and double bond positional specificities of the 16:0 desaturase. The studies demonstrated that substituting a major portion of the substrate binding channel of a Δ⁹-18:0 desaturase into the homologous position of a Δ⁶-16:0 desaturase converted its in vitro specificity to that of a Δ⁹-18:0 desaturase. This could also be accomplished by replacing one contact and four non-contact amino acids of the Δ⁶-16:0 desaturase with five amino acids of the Δ⁹-18:0 desaturase which occupy homologous positions. It was also shown that substituting bulky contact amino acid residues (isoleucine for proline at position 179 and phenylalanine for leucine at position 118) into the substrate binding channel of the Δ⁹-18:0 desaturase increased its preference for the 16:0-ACP substrate such that the in vitro 16:0-ACP activity became slightly more than two-fold greater than its remaining 18:0-ACP activity.

The ability to manipulate the chain length and double bond position specificities of desaturases has great potential with regard to generation and use of mutated native desaturases in the production of commercially useful products, such as vegetable oils rich in monounsaturated fatty acids. Such vegetable oils are important in human nutrition. In addition, because a double bond in an otherwise saturated carbon chain is readily susceptible to chemical modification, fatty acid chains having double bonds in unique positions produced by crop plants can be useful raw materials for industrial processes.

The earlier studies making use of molecular modeling and crystallographic data, while successful, were extremely time consuming and the in vitro activity of the altered enzymes was not directly correlated to the in vivo specificities of the altered enzymes. Those studies pointed out a need for a simplified and general method for readily producing mutants of desaturases which have altered and desirable chain length and double bond positional specificities.

### SUMMARY OF THE INVENTION

The present invention relates to a simple and general method for producing a mutant of a fatty acid desaturase, the original desaturase having an 18 carbon atom chain length substrate specificity, the mutant produced having substantially increased activity relative to the original desaturase towards fatty acid substrates with chains containing fewer than 18 carbons. The method involves inducing one or more mutations in the nucleic acid sequence encoding the original desaturase, transforming the mutated nucleic acid sequence under conditions for expression into a cell which normally requires a growth medium that is supplemented with unsaturated fatty acids in order to proliferate (i.e., an unsaturated fatty acid auxotroph cell), and then selecting for recipient cells which have received a mutant fatty acid desaturase with a specificity for shorter carbon atom chain length substrates. In a preferred embodiment, the mutated nucleic acid sequences are transformed into an E. coli unsaturated fatty acid auxotroph designated MH13. The cells are then grown in the absence of added unsaturated fatty acids to select for recipient MH13 cells which express mutated enzymes which are capable of producing sufficient unsaturated fatty acids in the cell to support growth, thereby overcoming the auxotrophy.

Another aspect of the present invention includes the mutants which are produced. Mutants of castor Δ⁹-18:0-ACP desaturase produced by the method arise from amino acid substitutions at specific residues. These mutants each have altered substrate chain length specificity, of 16- or fewer carbon atoms. Other embodiments of the present invention encompass the expression of the mutant desaturase molecules in individual cells and also in transgenic plants, for the production of specific fatty acid products.

Another aspect of the present invention is a method for specifically altering a function of a protein through directed mutagenesis. The method involves identifying candidate amino acid positions of the protein which, when mutated, are predicted to alter the function. A library of mutants of the protein which are produced by randomization of the amino acid at each candidate position, in combination with randomization of every other candidate position is generated, and mutants which exhibit the desired specific alteration of function are identified from the library. In a preferred embodiment, candidate amino acid positions are identified by a combination of methods, some examples being random mutagenesis, structural analysis of the protein, and sequence analysis of the protein. Examples of functions which the method can be used to alter include enzymatic functions, substrate specificity, binding functions, and structural functions. The method of the present invention is compared to the method of random mutagenesis in alteration of castor Δ⁹-18:0-ACP desaturase substrate chain length specificity.

### BRIEF DESCRIPTION OF THE DRAWINGS

FIG. 1 lists the amino acid sequence of mature castor enzyme (SEQ ID NO: 1), and the corresponding nucleic acid coding sequence (SEQ ID NO: 2).

FIG. 2 is a diagram illustrating the primers used for full positional randomization of site 117 of castor Δ⁹-18:0-ACP desaturase.

FIG. 3 is a diagram illustrating the primers used for the generation of a combinatorial library of castor Δ⁹-18:0-ACP desaturase with full positional randomization of positions 114, 117, 118, 179, 181, and 188.

### DETAILED DESCRIPTION OF THE INVENTION

The present invention is based on the use of a bacterial selection system for the selection of mutant desaturase molecules which have 18-carbon atom chain length substrate specificities prior to the introduction of the mutation and which have a 16 or fewer carbon atom chain length substrate specificity of as a result of the mutation. A preferred bacterial strain used in the selection system, E. coli MH13, is an unsaturated fatty acid auxotroph. MH13 normally requires a growth medium that is supplemented with unsaturated fatty acids in order to proliferate. Previous research (Cahoon, et al., (1996) J. Bacteriology 178:936-939 and Thompson, et al. (1991) Proc. Natl. Acad. Sci. USA 88:2578-2582) demonstrated that although 14:0 and 16:0 acyl-ACP desaturases were able to use in vivo pools of acyl-ACPs in E. coli to produce monounsaturated fatty acids, Δ⁹-18:0 acyl-ACP desaturases do not generate detectable amounts of monounsaturated fatty acids when expressed in E. coli. Thus, due to the substrate pools of saturated fatty acid substrates in E. coli, the Δ⁹-18:0 desaturase enzymes are not sufficiently active in the E. coli host cell and are thus not able to complement the deficiency in unsaturated fatty acid auxotrophs such as E. coli MH13. Desaturase enzymes which specifically utilize 18-carbon chain length substrates cannot complement the auxotrophy due to the low levels of such 18-carbon chain length substrates in the bacterial cell. However, introduction of a functional desaturase enzyme which has substantial activity towards fatty acid substrates with chains containing 16 or fewer carbons will complement this auxotrophy, allowing for the growth and proliferation of the bacteria in the absence of supplemental unsaturated fatty acids. These observations have been exploited as a selection system for identifying mutants of an 18-carbon specific fatty acid desaturase which have a substantially increased activity towards fatty acid substrates with chains containing 16 carbons or 14 carbons. While E. coli MH13 is a preferred host cell, one of skill in the art will recognize that other host cell types may be employed.

The present invention provides for a method of producing a mutant of a fatty acid desaturase, the mutant being characterized as having a specificity for shorter chain length fatty acid substrates compared to the original fatty acid desaturase. The method requires nucleic acid sequences encoding a fatty acid desaturase with 18 carbon atom chain length substrate specificity. To produce the mutant, mutations are induced in the nucleic acid sequence encoding the fatty acid desaturase. The mutated nucleic acid sequence is then transformed into the MH13 E. coli cells under conditions appropriate for expression of the mutated sequence. The transformed MH13 E. coli cells are then selected for the ability to grow in the absence of supplemental unsaturated fatty acids. Survival of a transformed MH13 E. coli indicates the acquisition of a mutant fatty acid desaturase which complements the fatty acid auxotrophy of MH13 because of its altered chain length specificity.

A mutant fatty acid desaturase identified by the above selection assay has a substantial increase in the activity towards fatty acid substrates with chains containing fewer than 18 carbons, relative to the original desaturase. A substantial increase in substrate specificity with respect to the original desaturase is one that produces sufficient accumulation of unsaturated fatty acids, which results from desaturation by the mutant desaturase, within an unsaturated fatty acid auxotroph host organism so as to support growth and proliferation of the host organism. Substantial increase in activity sufficient to support growth of the auxotroph host is at least three-fold higher than that of the non-mutagenized precursor desaturase. In a preferred embodiment, the increase in activity of the mutant desaturase is at least ten-fold higher than the non-mutagenized precursor desaturase.

The Exemplification section below details experiments where the method was used to identify mutants of castor Δ⁹-18:0-ACP desaturase with modified substrate specificities. One of skill in the art will recognize that the method is suitable for producing mutants of any fatty acid desaturase which has an 18 carbon atom chain length substrate specificity prior to mutagenesis. To do so requires only a nucleic acid sequence for the desaturase. Expression of the nucleic acid sequence results in the production of a mature fatty acid desaturase, and following mutagenesis of the nucleic acid sequence, those sequences which are mutated to cause the alteration in the chain length specificity of enzyme will be expressed and identified through the selection procedure.

The nucleic acid sequences having silent mutations which do not affect the amino acid sequence of the translated product would not be identified in the selection procedure. Nucleic acid sequences encoding a functional fatty acid desaturase, whose amino acid sequence varies from wild type, for example with conservative amino acid substitutions that do not affect function in regard to carbon chain length substrate specificity would also not be identified in the selection procedure. However, such mutated desaturases may be desirable when incorporating several different functional mutations into one mutant.

In preferred embodiments, the fatty acid desaturase is a plant fatty acid desaturase. There are two types of plant fatty acid desaturases, soluble (acyl-ACP desaturases), and integral membrane (acyl lipid desaturases), both of which are suitable for use in the present invention.

In one embodiment, the MH13 E. coli also express an exogenous plant ferredoxin. This can be accomplished by introduction of an expression vector containing sequences which encode plant ferredoxin (e.g. Arabaena vegetative ferredoxin), and the application of selective pressure to the resulting bacteria. The presence of plant ferredoxin, the redox partner of the plant desaturases, facilitates the function of the plant desaturase in E. coli. The presence of plant ferredoxin in the selection system allows for the selection of mutants with low specific activities towards fatty acids with 16 or fewer carbon atoms. Mutants which complement MH13 in the absence of plant ferredoxin are expected to have comparatively higher specific activities toward the shorter fatty acid substrates (Cahoon, et al. (1996)).

The selection system described above is most appropriate for use in selecting mutants with the desired substrate specificity from a population of heterogenous mutant fatty acid desaturase molecules. By transforming a population of mutated nucleic acid sequences, entire libraries of mutants can be screened for the ability complement the MH13 auxotrophy.

Any type of mutation which has the potential to result in a modified fatty acid desaturase protein product can be induced in the nucleic acid sequences. Logic based approaches of introducing amino acid substitutions into residues which interact with substrate are sound but can be very labor intensive and are mainly suited to cases in which structural information is available. Such methods have been successfully employed for modifying the chain length specificity of soluble desaturases, and for the introduction of double-bond versus hydroxyl group for the membrane class of enzymes (Cahoon et al., (1997); Shanklin, et al. (1998) Annu. Rev. Plant Physiol. Plant Mol. Biol. 49:611-641). Experiments described in the Exemplification section which follows demonstrate a form of site directed mutagenesis which specifically targets a particular codon, or codons, to produce amino acid randomization at specific position(s) within the protein product. These experiments also describe utilization of random mutagenesis, which has the potential to identify additional amino acids involved in substrate specificity. The use of random mutagenesis is perhaps the most powerful method because it does not rely on assumptions about which residues are important, assumptions which are based on structural information. The present method has the ability to identify substitution mutations at positions which are amino acid contact residues (positions which are located nearest the substrate within the binding channel) and also positions which affect the substrate specificity without directly contacting the substrate (Cahoon et al., (1997)).

Once mutated, the nucleic acid sequences are transformed into the MH13 cells. Transformation is preferably accomplished by electroporation, but alternative methods known to one of skill in the art can also be used.

Following transformation, the cells are selected for the presence of the mutant fatty acid desaturase. This is accomplished by growth on selective media (e.g. media lacking exogenously supplied unsaturated fatty acids). The media can be either solid or liquid. Using several rounds of selection, and/or varying or augmenting the selective pressures involved is also useful in increasing the number of mutants identified by the method.

The present invention is useful in the engineering of desaturase proteins with characteristic substrate chain length preferences. Such isoforms, when introduced into cells or organisms (e.g. agricultural crops) can be used to manipulate the physical properties and commercial uses of conventional plant oils. Cells and organisms which express these engineered desaturases are useful in the production of commercially useful products, such as vegetable oils rich in monounsaturated fatty acids, which have many potential uses, for example in human nutrition or as industrial chemicals.

The Exemplification section below details experiments where the above described method was used to identify mutants of castor Δ⁹-18:0-ACP desaturase which have modified substrate specificities. Mutations in the coding region which result in amino acid substitutions at position 114, 117, 118, 179, 181, or 188, and combined substitutions at positions 114 and 188, are described, along with the resulting altered specificity of these mutant proteins as compared to wild type. Mutations in the coding region of castor Δ⁹-18:0-ACP desaturase which produce a combination of amino acid substitutions at all six positions were also identified, the mutant proteins encoded being referred to as com2, com3, com4, com9, and com10. Table 3 lists the amino acid substitutions and the specific activities of these mutant proteins for the different substrates.

All five mutant proteins listed in Table 3 have the amino acid substitutions T117R and G188L, in combination with various substitutions at the remaining four positions. The fact that these two mutations are the optimal changes at their respective positions for reducing chain length specificity suggests that they are likely the primary determinants of the altered specificity in com2. The observation that several other mutants containing this pair of mutations have lower specific activity suggests that the combination of mutations at the remaining four randomized sites can also affect the specific activity of the mutants. This conservation suggests that the substitutions T117R and G118L are responsible for the change in substrate specificity of the five mutants in Table 3. A mutant desaturase with the combination of T117R and G118L substitutions is expected to have enhanced activity for one or more substrates with 16 or fewer carbons.

Another aspect of the present invention is a mutant castor Δ⁹-18:0-ACP desaturase which has a subset of the amino acid substitutions of a mutant protein listed in Table 3. Such a mutant is expected to also have altered activity towards the different substrates. The present invention encompasses mutant castor Δ⁹-18:0-ACP desaturase proteins which have between 1 and 6 of the amino acid substitutions of the com2 mutant, in any possible combination. In a preferred embodiment, the combination includes at least three of the amino acid substitutions of the com2 mutant. Preferably two of these amino acid substitutions are T117R and G188L. In addition, the present invention is intended to encompass mutant Δ⁹-18:0-ACP desaturase proteins which have between 1 and 6 of the amino acid substitutions of the com3 mutant, the com4 mutant, the com9 mutant, or the com10 mutant, respectively, in any possible combination. In a preferred embodiment, the combination includes at least three of the amino acid substitutions, of the mutant. Preferably two of these substitutions are T117R and G188L. Also included in the present invention are mutants which have the above listed amino acid substitutions, and combinations thereof, in combination with any other amino acid substitutions, insertions or deletions. These additional substitutions, insertions or deletion, may be silent (e.g. do not affect function of the enzyme) or may further alter enzyme function.

The above listed amino acid substitutions made at the analogous positions in other ACP-desaturases, especially in 18:0-ACP desaturases, and preferably in Δ⁹-18:0-ACP desaturases, are predicted to have the analogous effects on substrate specificity in these proteins as in the disclosed desaturase mutants.

Nucleic acid sequences which encode the mutant proteins described above, can be inserted into a DNA expression vector, which can then be used to express the mutant proteins in cells. Expression vectors which function in either or both prokaryotic and eukaryotic cells exist and are known to those of skill in the art. The appropriate expression vectors are introduced into either prokaryotic cells, (e.g. bacteria) or eukaryotic cells (e.g. animal cells or plant cells) under conditions appropriate for expression of their coding sequences. Plant cells which express the mutant proteins can be used to produce transgenic plants which express the mutant proteins, and which produce the corresponding fatty acid products of the desaturases.

Another aspect of the present invention is a method for specifically altering a function of a protein through directed mutagenesis. Upon determination of the specific function which is to be altered, candidate amino acid positions of the protein which are predicted to alter the function when mutated, are identified. Several methods for identifying candidate positions are described below. A library of mutants of the protein are generated by randomization of the amino acid encoded at each candidate position, in combination with randomization of every other candidate position within each mutant. This is generally accomplished by generating a library of mutant nucleic acid sequences which encode the mutant proteins through simultaneous randomization of the codons for each candidate position, in combination with randomization of every other candidate position within each mutant. Mutant proteins, encoded by the mutated nucleic acid sequences, which exhibit the desired alteration of function are then identified from the library.

A wide variety of functions are performed by proteins. Some proteins function as enzymes which catalyze reactions (e.g. catabolic, anabolic), some proteins function as binding proteins (e.g. ligand binding receptors, antibodies, adapter proteins), some proteins function as structural proteins (e.g. extracellular matrix proteins). The present invention is useful for altering any given function of any given protein. Because many proteins have more than one function, the specific function which is to be altered must first be determined. Often the functions of a multifunctional protein are independent of one another, allowing one function to be altered without affecting the other function(s) of the protein. In other cases, the functions are interlinked or interdependent, making alteration of a single function more complex. Alteration of a function is broadly defined herein as including any directed change in the function. Such changes include, without limitation, optimization of a function, (e.g. increasing the specific activity of an enzyme, increasing the binding affinity of a binding protein, increasing the integrity or stability of a protein's structure), redirection of a functional property of a protein (e.g. modifying the substrate specificity of an enzyme, modifying the binding specificity of a binding protein, modifying a structural component of a protein), and reduction (e.g. abolishing) of a function. Complete alteration of a designated function may necessarily be achieved in stages through sequential alteration of individual components of the function, producing a series of intermediate mutants, the entire process culminating in the generation of a final optimal mutant. Therefore, the process of altering a function of a protein as described herein, is intended to include optimizing, redirecting, or reducing a function of a previously altered protein.

Candidate positions include the positions of amino acids of the wild type protein which are involved (either directly or indirectly) in the function. Importantly, an indication of involvement in the function is all that is required for selection of candidate positions. The direction an individual mutation has or is expected to have on the function is unimportant in the identification of candidate positions. For instance, residues which when mutated individually, result in a decrease of the function, may be identified as candidates. Examples of amino acids which are directly involved with function include, without limitation, residues which make contact with other molecules involved in the function (e.g. substrate or ligand), and also residues which line or define binding sites. Examples of amino acids which have indirect involvement include, without limitation, residues which influence those directly involved residues, such as residues adjacent or near directly involved residues. Proximity need not be limited to primary sequence, but may be from secondary or tertiary structure relationships. In addition, residues may be located near directly involved residues due to the formation of inter- or intra-molecular complexes. The influence that indirectly involved amino acids can have may be steric effects, chemical effects, or a combination of effects. Indirectly involved amino acids also include residues which participate in defining an element of the protein structure which is crucial for the function (e.g. the necessary conformation of a protein).

Candidate positions also include positions of amino acids which are not significantly involved (directly or indirectly) in the function of the wild type protein, but which assume a role (direct or indirect) in function through mutagenesis. The term wild type is used herein to refer to the original sequence of a protein prior to mutagenesis, the term being inclusive of previously altered sequences. Mutagenesis which confers a role in function to a previously uninvolved residue commonly involves the substitution of another amino acid at that particular position. However, a new involvement in function may also be conferred to a position by mutagenesis at another position.

The more information one has regarding the protein, the function of the protein which is to be altered, and the residues which participate in the function, the more productively one can go about altering the function. Candidate amino acid positions of the protein are identified by any number of means. Without limitation, such means include, random mutagenesis of the nucleic acid sequences encoding the protein, structural analysis of the protein, and sequence analysis of the protein, often coupled with comparison to related proteins. Methods for identification of candidate positions may be performed with the naturally occurring protein, or alternatively with a mutant version of the protein. In addition, analysis of related proteins (e.g. sequence analysis, structural analysis, mutagenesis) may indicate analogous candidate positions within the protein of interest which are likely involved in the function to be altered. The term related proteins as used herein includes different isomers of a protein, different phenotypes of a protein (e.g. naturally occurring mutants of the same protein), and any other proteins or fragments thereof which have significant homology to the protein whose specific function is to be altered.

Random mutagenesis coupled with screening for loss or change of function mutants can identify amino acid positions which are crucial for function of the wild type protein. Random mutagenesis coupled with screening for gain or enhancement of function mutants can identify these crucial positions as well as positions which only minimally participate in wild type function, but have gained an increased role through mutagenesis.

Structural analysis of a protein is also a very powerful tool with which to identify candidate residues. Structural information can be obtained from X-ray crystallography, or from other methods such as nuclear magnetic resonance. Often the structure of a protein performing of the function (e.g. an enzyme bound to substrate or an inhibitor, or a binding protein bound to ligand) provides a significant amount of information regarding the amino acid positions involved in the function.

Preferably, a combination of methods are employed to identify candidate amino acid positions. In a preferred embodiment, all available means are employed to ensure identification of as many candidate positions as possible.

Once the candidate positions are identified, a library of mutant proteins which have every candidate position within each mutant randomly substituted with 1 of 20 possible amino acids. This method of substituting 1 of the 20 possible amino acids at a specific position within a protein is referred to herein as randomization of the amino acid. Randomization of the amino acid encoded at each and every candidate codon within an individual particular protein is referred to herein as combinatorial full positional randomization. A library of mutant proteins resulting from combinatorial full positional randomization is most easily produced by generating a nucleic acid library of mutated coding sequences of the protein, which have 1 of 20 possible amino acid encoded at every candidate position within. Because this type of mutagenesis allows for the insertion of a codon for the wild type residue, as well as the other 19 residues, at each candidate position, this produces the widest possible variety of mutation combinations. Combinatorial full positional randomization of codons can be accomplished by a variety of methods. One such method is the use of overlap-extension PCR to replace all codons for candidate position amino acids with NNK or NNN. The process of overlap-extension PCR has been used to simultaneously introduce at least nine independent mutations into a particular coding sequence.

In another embodiment, a subset of one or more of the candidate positions are incompletely randomized, while the other candidate positions are fully randomized. That is to say, fewer than the 20 possible amino acids are introduced at one or more designated candidate positions, to more specifically direct the mutagenesis. This is accomplished by randomly replacing the subset of candidate position codons of the nucleic acid sequence which encode the protein, with codons that encode the desired subset of amino acids, while introducing codons which encode all 20 amino acids at the other candidate positions.

Once the library is generated, mutant proteins which exhibit the desired altered function are identified. This is most efficiently accomplished by using a functional selection process. The mutated nucleic acid sequences are expressed individually, preferably by individual introduction into a single celled organism under conditions appropriate for expression. Once expressed, the mutant proteins which exhibit the desired function may be selected by their function (e.g. a complementation assay). Alternatively, populations of mutants generated can be screened for the desired altered function (e.g. by a rapid screening process). Each mutant generated can also be individually assayed for the desired altered function.

Experiments detailed in the Exemplification section which follows were performed to modify the substrate specificity and specific activity of castor Δ⁹-18:0-ACP desaturase. Structural analysis of the protein was combined with random mutagenesis to identify candidate residues.

Random mutagenesis was used to identify candidate residues of the desaturase by a functional assay. Theoretically, this method of identification has the potential to identify residues which may or may not line the substrate binding cavity, they are simply identified by a functional assay, thus this method of identifying residues likely to participate in function is applicable to both enzymes for which a structure is known, and enzymes for which a structure is unknown. All relevant knowledge should be included in compiling the list of candidate amino acid positions to be randomized.

One distinguishing feature of the present invention is that combinatorial full positional randomization is performed simultaneously on all candidate positions which are identified. Previous approaches to directed mutagenesis to specifically alter a function of a protein have used a multistep approach, where one residue is mutated, the mutant is characterized, and then that mutant is subjected to another round of single position mutagenesis. This standard approach results in each subsequently produced mutant carrying over specific mutations from the last mutant product. Thus, each subsequent mutant identified is necessarily constrained by properties inherited from the mutant from which it is generated, thus limiting the direction the mutagenesis may take to achieve the desired function. By eliminating this limitation, the method of the present invention generates a wider variety of mutants which demonstrate the desired activity, from which one can select an optimal mutant.

### Exemplification

### Section I: Preliminary Studies

A mutagenesis and selection approach was employed to identify amino acid substitution mutations in plant fatty acid desaturases which modify substrate specificity. Acyl-ACP desaturases are functionally active when expressed in E. coli. Δ⁹-18:0-ACP desaturases are unable to alter the fatty acid profile of E. coli due to a lack of appropriate substrate (Thompson et al., (1991)). However, desaturases with 16:0 or 14:0 specificity were shown to alter the fatty acid profile of E. coli (Cahoon, et al. (1996)). Thus, 18:0 desaturases cannot complement the E. coli mutant MH13, an unsaturated fatty acid auxotroph, but desaturases with specificities with 16 or fewer carbons are able to complement this auxotrophy. Thus, the MH13 E. coli strain was used to select for mutants of an 18-carbon desaturase which can utilize 16- or 14-carbon substrates in a complementation assay.

To facilitate the function of a plant acyl-ACP desaturase in E. coli, an expression vector containing the gene for plant-type ferredoxin, the redox partner of the plant desaturase, was transformed into the MH13 E. coli and maintained under selective pressure. These cells, MH13(pACYC/LacAnFd) were used in the following experiments.

The nucleic acid sequence for castor Δ⁹-18:0-ACP desaturase was subjected to one of two types of mutagenesis, site directed or random mutagenesis, prior to introduction into the MH13 cells. PCR was used in site directed mutagenesis to randomize a targeted codon corresponding to a specified residue in the amino acid sequence of the castor Δ⁹-18:0-ACP desaturase. Target codons corresponding to Met 114, Leu 118, Pro 179, and Gly 188 were each subjected to independent randomization. Previous studies (Cahoon, et al. (1997)) had indicated that these residues are located adjacent to the substrate binding cavity and that replacing some of those amino acids in the T. alata Δ⁶-16:0 desaturase or in the castor Δ⁹-18:0 desaturase with bulkier or less bulky amino acids could affect substrate specificity in vitro. The methods of the present invention allowed for an unbiased substitution of all 20 amino acids into these positions but required that the mutation have an affect on the in vivo substrate specificity of the desaturase. The mutagenesis reactions yielded four populations, each one comprising a library of coding sequences with substitution mutations consisting of all 20 potential amino acids at the designated mutation site.

To examine whether mutations in additional contact and/or non-contact residues could alter the in vivo substrate specificity of the castor Δ⁹-18:0-ACP desaturase, a totally unbiased approach using random mutagenesis was performed on the sequences encoding castor Δ⁹-18:0-ACP desaturase by single gene DNA shuffling.

MH13(pACYC/LacAnFd) were transformed with the resulting libraries of mutated 18:0-ACP desaturase, under conditions appropriate for expression, and then selected for expression of a mutant with the ability to complement the unsaturated fatty acid auxotrophy, by growth in the absence of supplemental unsaturated fatty acid. To confer survival under the selective conditions, a mutant desaturase would necessarily have an altered substrate chain length specificity of 16, 14 or fewer carbons. The selection for site directed mutants was performed in either liquid media or on agar plates. The selection for randomly generated mutants was performed on agar plates. Growth in liquid media involved several rounds of dilution and re-growth to enrich for mutations that resulted in the best complementation.

In a variation on the site directed mutagenesis, mutants were selected from a library encoding all 400 possible combinations of amino acids at position 188 and 114, two adjacent contact residues within the substrate binding channel. This was achieved by excising a restriction fragment from the open reading frame of the library encoding all possible amino acids at position 188 and inserting this fragment into the equivalent plasmid population randomized for position 114. Using this method, mutant M114I-G188L was identified in the selection procedure. The coding sequences of the selected desaturases were sequenced to identify the specific mutations which conferred complementation to the fatty acid auxotrophy. The substrate specificities of the identified mutants were determined by in vitro enzyme assays (Cahoon et al., (1997)). Table 1 lists the identified mutations and the altered chain length substrate specificity conferred.

TABLE 1

| | | | Fold change in specificity | |
|---|---|---|---|---|
| | Mutagenesis Method | | with respect to wt | |
| Position | Directed | Random | 16:18 | 14:18 |
| Met 114 | Ile (16) | Ile (16) | 6 | |
| Met 114 | Phe (14)/ | | Phe 7 | 490 |
| | Tyr (14) | | | |
| Thr 117 | | Ile (16) | not determined | |
| Leu 118 | Phe (16)/ | Phe (16)/ | Tyr 130 | |
| | Tyr (16) | Met (16) | | |
| Pro 179 | Ile (16) | Leu (14) | 20 | |
| Thr 181 | | Ile (16) | not determined | |
| Gly 188 | Leu (16) | | 740 | |
| Met 114/ | M114I/ | | 1410 | |
| Gly 188 | G188L (16) | | | |
| Table 1. Mutations obtained by mutagenesis/selection numbers in parentheses represent the chain length specificity that is most enhanced with respect to the wt castor Δ⁹-18:0 desaturase activity. Shown are also the fold change in specificity ratios where known. For instance, if the activity with respect to 16 carbon was increased by 10-fold, and the activity with respect to 18:0 was decreased by 5-fold, the | | | | |
| # “Fold change in specificity with respect to wt” for 16:18 would be 50. | | | | |

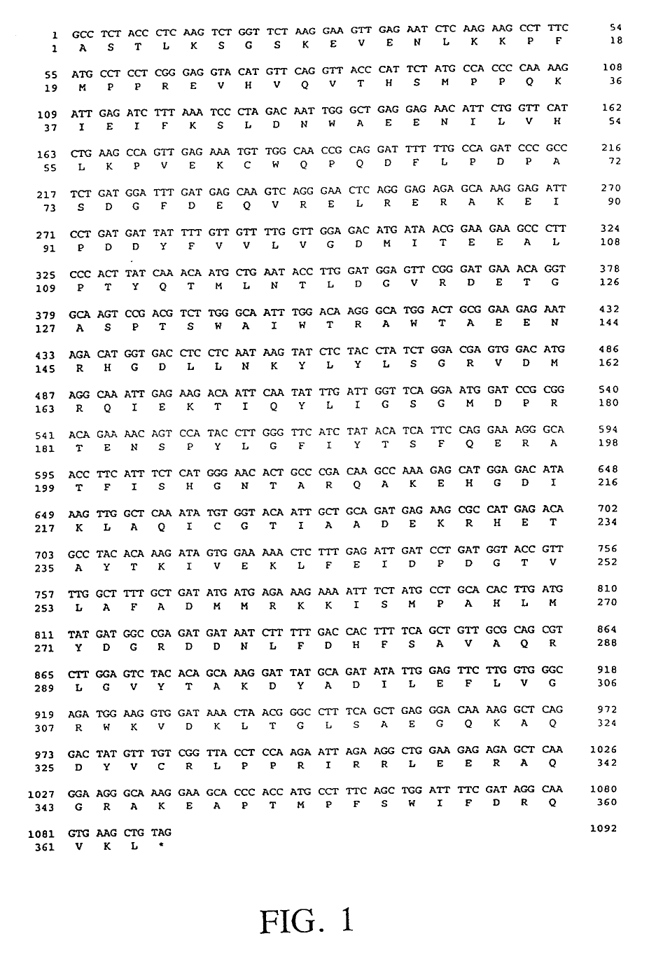

The designated amino acid positions above correspond to the mature castor enzyme as defined in Lindqvist et al., EMBO J. 15: 4081-4092 (1996), the sequence of which is listed in FIG. 1 (SEQ ID NO: 1).

While the use of structure-guided (i.e., directed) mutagenesis of residues M114, L118, P170 and G188 was effective for the identification of seven mutants with substrate specificities of 16 or fewer carbon fatty acids, the method relied on the appropriate choice of target residues for mutagenesis. It is well documented that residues that affect substrate specificity fall into two broad classes, direct and indirect. Thus, random mutagenesis selection provides a bias-free method for the identification of changes that result in increased specificity for shorter acyl chains. Through random mutagenesis and selection of the present invention, five amino acid positions were identified, three at sites that were also targets for the structure-guided mutagenesis and two new sites, T117 and T181.

The naturally occurring 16:0-ACP desaturases from Milkweed and Doxantha have very poor activities when assayed in vitro (31 and 3 nM/min/mg, respectively). However, the selected mutant G188L has an activity of (175 nM/min/mg) much closer to that of the parental wild type castor Δ⁹-18:0-ACP desaturase with its 18:0-ACP substrate.

To test whether the altered enzymes identified in the selection assay would result in the accumulation of unusual fatty acids when expressed in plants, the G188L mutant was introduced into Arabidopsis thaliana (fab1 background) using a napin promoter to drive expression. The first generation of G188L transgenics (T1) produced seeds which contained approximately 10% of fatty acids modified by the introduced desaturase. Because Ti seeds are heterozygous it is anticipated the levels of desired fatty acids will increase in the homozygous T2 plants. These results suggest that mutants derived from castor Δ⁹-18:0-ACP desaturase may be useful for future metabolic engineering of oil crops.

Materials and Methods for Section I

Cell lines. The E. coli unsaturated fatty acid auxotroph MH13 mutant of E. coli K12 (Henry, M. F., Ph.D. Thesis, University of Illinois, Urbana-Champaign (1992)) is a fadR::Tn5 mutant of cell line DC308 (Clark et al., (1983) Biochemistry 22:5897-5902) which was constructed by phage P1 transduction from strain RS3069 (Simons et al., (1980) J. Bacteriol. 142:621-632). MH13 requires a medium supplemented with unsaturated fatty acids at all growth temperatures due to a temperature-sensitive lesion in fabA and transposon disruption of fadR. An XbaI/EcoRI fragment from a pET9d expression plasmid containing the coding sequence of Anabaena vegetative ferredoxin (Fd) (Cheng et al., (1995) Arch. Biochem. Biophys. 316:619-634) was inserted into the corresponding sites of pLac3d to generate the plasmid pLacAnFd. pLac3d is analogous to pET3d except that the T7 RNA polymerase promoter has been replaced with the lacUV5 promoter of E. coli RNA polymerase as described previously (Cahoon et al., (1996)). A BglII/HindIII fragment from pLacAnFd was then inserted into the BamHI/HindIII sites of pACYC184. This construct (pACYC/LacAnFd) was then introduced into MH13 cells by electroporation.

Complementation Analysis/Selection.

The E. coli MH13 strain harboring pACYC/LacAnFd was used as a host for expression of acyl-ACP desaturases. For these studies, the coding sequence of wild type and mutant mature acyl-ACP desaturases were inserted into pLac3d. Cells were transformed with the resulting plasmid constructs and were then grown on plates or in liquid broth containing Luria-Bertani (LB) media with ampicillin (100 μg/ml), chloramphenicol (35 μg/ml), and kanamycin (40 μg/ml) selection. For non-selective growth, plates were supplemented with the fatty acid oleic acid solubilized in Tergitol NP-40 (Sigma) with final concentrations of 250 μg/ml oleic acid and 2% (v/v) Tergitol. Liquid broth was supplemented with oleic acid (solubilized in Tergitol NP-40) at a final concentration of 100 μg/ml. Oleic acid was initially prepared as 1000x stock solution in ethanol and solubilized in melted Tergitol, prior to addition to the media. Media used to test for complementation did not contain added oleic acid, and IPTG was added at a concentration of 0.4 mM to induce expression of acyl-ACP desaturase.

Transformation.

Transformation was conducted by electroporation using a 50 μl aliquot of competent MH13 cells harboring pACYC/LacAnFd and 0.1 to 0.5 μg of expression plasmid for a given acyl-ACP desaturase. Following electroporation, cells were resuspended in 500 μl of LB media and shaken (250 rpm) at 37° C. for 45 min to 1 h. Cells were then plated on media as described above. Alternatively, a 75 μl aliquot of the transformed cells was added to 25 ml of LB media containing IPTG and antibiotics at concentrations described above. These cells were then maintained with shaking at 30° or 37° C.

Electrocompetent MH13 (pACYC/LacAnFd) were prepared by growing a culture from a single colony in low-salt LB media (10 mg/ml Bacto tryptone, 5 mg/ml yeast extract, and 5 mg/ml sodium chloride) containing kanamycin (40 μg/ml) and chloramphenicol (35 μg/ml) and supplemented with oleic acid (100 μg/ml) and 2% Tergitol (v/v). Cells were prepared for transformation and electroporated as described in the BioRad protocol for high efficiency electro-transformation of E. coli.

Mutagenesis.

Two methods were used for mutagenesis. The first, site directed mutagenesis, randomized a target residue at a specific location in the amino acid sequence of the castor Δ⁹-18:0-ACP desaturase. Four target residues were chosen: Met 114, Leu 118, Pro 179, and Gly 188. PCR was used to generate four populations of DNA. Each population consisted of sequences encoding castor Δ⁹-18:0-ACP desaturase with a randomized codon for residue 114, 118, 179, or 188. Each of the four populations was generated using PCR site directed mutagenesis to produce DNA products having equimolar proportions of each of the four nucleotides at each position of the target codon. For each of the four randomized products, an oligonucleotide primer was synthesized which hybridized to sequences adjacent to the target codon, and contained a randomized codon in place of the target codon sequences, the primer population containing equimolar proportions of each of the four nucleotides G, A, T, and C at the three positions within the replacement codon. This primer was used in conjunction with a primer homologous to the 5′ terminus of the gene to amplify the gene segment between the two primer binding sites. A second overlapping fragment was then synthesized using PCR to amplify the remainder of the respective coding sequences of the four PCR reaction products. The fragments were then incorporated into larger gene fragments using overlap extension polymerase chain reaction (Ho et al., (1989) Gene 77:51-59). The gene fragments containing the randomized target codons were inserted into pLac3.

The second mutagenesis method introduced random mutations into the coding region sequence by digesting the castor Δ⁹-18:0-ACP desaturase coding region with DNase, and reassembling using PCR (W. P. Stemmer, (1994) Proc. Natl. Acad. Sci. USA 91:10747-10751). The entire coding region was reinserted into pLac3 to make a library of pLac3 castor Δ⁹-18:0-ACP desaturase genes with random mutations throughout the coding region.

### Section II: Comparison of Full Positional Randomization with Other Methods

Single Position Mutagenesis

Four amino acid positions in the castor Δ⁹-18:0-ACP desaturase were identified by structural analysis (deductive reasoning by examination of a crystal structural model) as likely to participate in substrate specificity: 114, 118, 179, and 188. Independently, five positions were identified by random mutagenesis as likely to participate in substrate specificity: 114, 118, 179, 117, and 181. Together, this yielded a total of six positions identified as likely to participate in substrate specificity: 114, 117, 118, 179, 181, 188. To identify which of the 20 possible amino acids inserted at a target position would produce a mutant with the highest activity on 14 and 16 carbon substrates, libraries of mutants were generated by randomization of one of these positions at a time. Randomization resulted in insertion of 1 of all 20 possible amino acids at the target position. Six libraries were generated, one for each target position. These libraries were introduced into MH13 bacteria to select for mutants with enhanced substrate specificities of 14 or 16 carbon chain length. Complementation of MH13, the unsaturated fatty acid auxotroph of E. coli, requires that the desaturase have a substrate specificity for 14 or 16 carbon chain length (or perhaps fewer) fatty acids. Recipient colonies which were able to grow under selective conditions were isolated. The mutant desaturase was purified from a crude lysate of a culture of the colony, and the specific activities for 14 and 16 carbon chain length substrates were determined for selected mutants by assay of the protein. For some mutants the specific activities of the enzyme were determined for 14, 16 and 18 carbon chain length fatty acyl ACP substrates.

Mutants produced from the two types of mutagenesis were identified and compared. Table 2 lists the most active mutants identified by the two methods. Table 3 compares the activities of mutants at position 117 and mutants at position 181, which were identified using both methods of mutagenesis. Mutants identified from the libraries of target position randomized mutants (full positional randomization) demonstrated the higher specific activities for 14 and 16 carbon substrates than did the mutants which were identified by random mutagenesis. Selection of mutants from a library produced by random mutagenesis only once (L118F) produced the same, most active mutant identified by the full positional randomization method. Other selected mutants which resulted from random mutagenesis had catalytic rates inferior to the rates of mutants produced by full positional randomization. The reason for this is that random mutagenic process used could only generate between 4 and 7 amino acid substitutions per position. With respect to the six positions, at five of the positions, when offered all 19 substitutions by full positional randomization, a mutant enzyme with higher specific activity was obtained, and in the sixth case an identical enzyme having equivalent activity was obtained by both methods. This demonstrated that the greater number of substitutions tested for a single position facilitated an increased probability of a higher increase in specific activity for a particular substrate.

TABLE 2

| | Random Point Mutagenesis | | | | Full Positional Randomization² | | | |
|---|---|---|---|---|---|---|---|---|
| | Act³ (nM/min./mg) | | | | Act³ (nM/min./mg) | | | |
| Site⁴ | Sub⁵ | 14 | 16 | 18 | Sub⁵ | 14 | 16 | 18 |
| Met 114 | Ile | 3 | 22 | 260 | Ile | 3 | 22 | 260 |
| Met 114 | | | | | Phe/Tyr⁶ | 5 | 0.8 | 7 |
| Thr 117 | Ile | 0.7 | 5 | 35 | Arg⁶ | 3 | 30 | 332 |
| Leu 118 | Phe | 0.5 | 42 | 270 | Phe | 0.5 | 42 | 270 |
| Pro 179 | Leu | 15 | 22 | 206 | Ile⁶ | 18 | 78 | 270 |
| Thr 181 | Ile | 0.7 | 5 | 196 | Phe⁶ | 5 | 64 | 198 |

| | | | | | |
|---|---|---|---|---|---|
| Gly 188 | None Identified | Leu⁶ | 11 | 173 | 19 |

Key:
¹Random point mutagenesis of the castor Δ⁹-18:0-ACP desaturase.
²Full positional randomization using a primer encoding NNK to give 32-fold degeneracy with codons encoding all 20 amino acids.
³Specific activity measured using ¹⁴C-argentation thin layer chromatography assay in conjunction with phosphor imaging quantitation.
⁴Site of the amino acid position with respect to the mature castor open reading frame see following sheet for number definition.
⁵Substituted amino acid, in standard three letter format.
⁶Underlined amino acids identify substitutions that were not attainable by point mutagenesis.

TABLE 3

| | | | Act¹ (nM/min./mg) | | |
|---|---|---|---|---|---|
| Site² | Method | Sub⁴ | 14 | 16 | 18 |
| Wt | | None | 0.8 | 12 | 820 |
| Thr 117 | RPM³ | Ile | 0.7 | 5 | 35 |
| | FPR⁴ | Arg⁵ | 3 | 30 | 332 |
| | FPR | Val | 1 | 0.7 | 35 |
| | FPR | Lys | 3 | 2.6 | 93 |
| Thr 181 | RPM | Ile | 0.7 | 5 | 196 |
| | FPR | Phe | 5 | 64 | 198 |
| | FPR | Trp | 25 | 27 | 12 |
| | FPR | Leu | 2.4 | 42 | 181 |
| | FPR | Met | 3 | 15 | 479 |

Key:
¹Specific activity measured using ¹⁴C-argentation thin layer chromatography assay in conjunction with phosphor imaging quantitation.
²Site of the amino acid position with respect to the mature castor open reading frame see following sheet for number definition.
³RPM randomized point mutagenesis of the castor Δ⁹-18:0-ACP desaturase
⁴FPR: Full positional randomization (using a primer encoding NNK to give 32-fold degeneracy with codons encoding all 20 amino acids).
⁵Underlined amino acids identify substitutions that were not attainable by single point mutagenesis.

Combinatorial Full Positional Randomization

In an effort to produce a mutant desaturase protein which had enhanced activity for 14 and 16 carbon substrates, a library of mutants were generated by randomizing all six target sites, 114, 117, 118, 179, 179, and 188, simultaneously. This procedure is termed combinatorial full positional randomization. The library was introduced into MH13, which were then plated on selective (unsupplemented) media. A sample set of 19 colonies which grew on the selective media were picked. The plasmid DNA was isolated from each colony and used to re-transform the MH13 to confirm that the plasmids encoded a modified desaturase. Plasmids from each of the selected colonies were purified and the DNA sequence of each selected mutant encoded was determined.

Conceptual translation of the DNA sequences indicated that all 19 mutants had distinct combinations of amino acids at the six target sites. All 19 mutant desaturase enzymes were produced and purified and subjected to in vitro enzyme assays. Table 4 lists five of the mutants produced and their specific activities for the different substrates.

TABLE 4
Combinatorial Mutants Containing T117R and G188L

| | Position | | | | | | Activity¹ | | |
|---|---|---|---|---|---|---|---|---|---|
| | 114 | 117 | 118 | 179 | 181 | 188 | 14 | 16 | 18 |
| Wild Type | M | T | L | P | T | G | 0.8 | 11.5 | 820 |
| Mutants | | | | | | | | | |
| com2 | A | R | G | V | V | L | 59 | 270 | 420 |
| com3 | Q | R | P | V | D | L | 0 | 13 | 3 |
| com4 | T | R | A | L | S | L | 13 | 132 | 12 |
| com9 | V | R | G | S | C | L | 1.9 | 42 | nd² |
| com10 | Y | R | P | A | F | L | 2 | 22 | nd² |

¹Activity (nm/min/mg) determined with 3 fatty acid ACP substrates
²nd = not determined

The mutation having the highest specific activities for the tested substrates was com2. This mutant had far higher specific activity for 14 and 16 carbon substrates than did any of the single position mutants, exhibiting 74 and 23 times wild type activities for the respective substrates (Table 5).

TABLE 5
Comparison of Combinatorial Full Positional Randomization
with Other Methods¹

| Position | FPR | 14 | 16 | RPM | 14 | 16 |
|---|---|---|---|---|---|---|
| M114 | Phe | 6 | 0.1 | Ile | 4 | 2 |
| L118 | Phe | 1 | 4 | Phe | 1 | 4 |
| P179 | Ile | 23 | 7 | Leu | 19 | 2 |
| G188 | Leu | 14 | 15 | — | — | — |
| T117 | Arg | 4 | 3 | Leu | 1 | 0.5 |
| T181 | Trp | 31 | 2 | Leu | 1 | 4 |
| Com2 | | 74 | 23 | | | |
| Com2 | M114A | T117R | L118G | P179V | T181V | 0188L |

¹All activities are reported as the fold change in activity compared to the wild type castor Δ⁹-18:0-ACP desaturase activity for that chain length (i.e., 0.8 and 11.5 nm/min/mg protein for 14 and 16 carbon atom substrates, respectively.
Underlined residues are those yielding the highest increase when tested for activity in single mutants.

Two of the amino acid substitutions in com2, T117R, and G188L, were determined to be the optimal substitutions for those positions by the independent randomization studies described above. This correlation indicates that the increase in specificity of the com2 mutant is due to the substitutions at these two residues. Notably, neither of these changes were available via point mutagenesis. As indicated in Table 4, five of the 19 combinatorial mutants analyzed contained these two specific mutations, T117R and G188L, but contained different substitutions at the other four positions. These five mutants exhibited various changes in specific activity for the two substrates. Thus substitutions at positions 114, 118, 179 and 181 had a profound effect on the influence of the changes at positions 117 and 188. Therefore, the combination of substitutions at the other four sites could either accentuate the positive effects that T117R and G188L had on activity, or alternatively, could block the effect. Increasing the number of possible combinations of amino acids at all positions identified as affecting the substrate specificity (e.g. when substituted individually), including amino acid substitutions which are sub-optimal for affecting the substrate specificity individually, yields mutants with optimal activities.

Indeed, the accommodation of mutations which produce positive changes without in turn causing negative changes is likely of great importance for obtaining optimal performance. One could, in a sense, look at amino acids as molecular shims in the structure, the more shims of different sizes and properties that can be used to modulate the structure, the higher the likelihood that any particular structure will have optimal activity. Thus the approach of identifying as many positions as possible which might affect a particular property of the protein, and then presenting as many combinations at each of those positions as possible, coupled with an appropriate screening process, will identify a mutant protein which has optimal activity. For the positions mutated in this example, point mutagenesis could result in a limited number of amino acid substitutions: 6 at M114; 5 at T117; 5 at L118; 6 at P179; 6 at T181; and 5 at G188. Thus the total combinatorial number would be 6³×5³=27,000 unique mutants. If all 20 combinations were permissible as in the combinatorial full positional randomization method, that number would rise to 19⁶=47,045,881, or 1742-fold more combinations from which to select the optimal mutant for the particular trait. Since subtle changes can dramatically affect the activity of a protein, methods which result in more rather than fewer mutations of positions of amino acids shown to affect catalytic rates, will always produce equal or superior results to methods employing the more restrictive point mutagenesis.

Materials and Methods For Section II are the same as those used for Section I, unless as otherwise described below.

Full positional randomization.

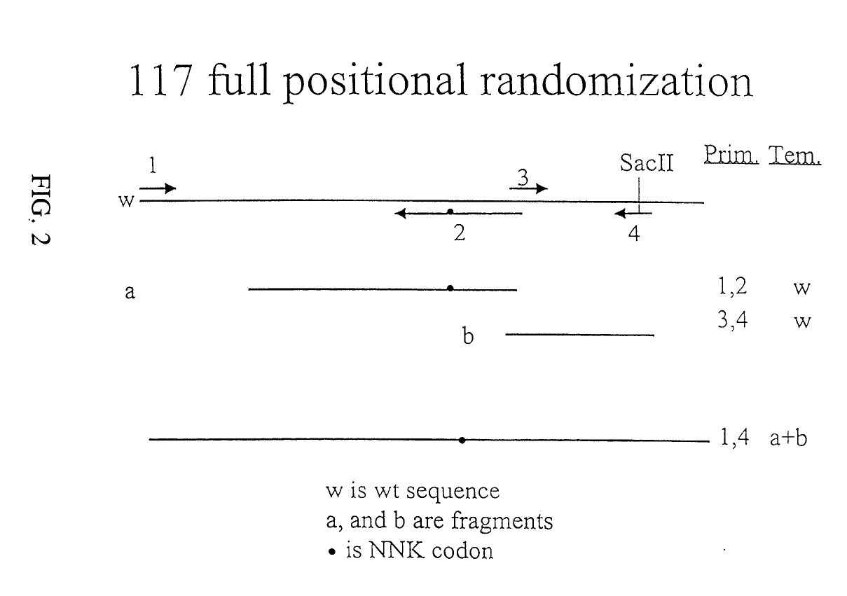

Castor-Δ⁹-18:0-ACP desaturase was subjected to mutagenesis prior to introduction into the MH13 cells. PCR was used in site directed mutagenesis to randomize the three residues comprising a codon corresponding to a specified residue in the amino acid sequence of the castor Δ⁹-18:0-ACP desaturase. Target codons corresponding to Met 114, Thr 117, Leu 118, Pro 179, Thr 181 and Gly 188 were each subjected to independent randomization. Because these residues are located adjacent to the substrate-binding cavity, amino acid substitutions at these positions are considered highly likely to affect substrate specificity. These mutagenesis reactions yielded four populations, each one comprising a library of coding sequences with substitution mutations consisting of all 20 potential amino acids at the designated mutation site. An example for the introduction of all possible amino acid substitutions at position 117 by overlap extension PCR is diagrammed in FIG. 2. Primers used were 1: GTGAGCGGATAACAATTTCACACAG TCTAGAAAT (SEQ ID NO: 3), sequence flanking the unique XbaI site at the 5′ end of the open reading frame; 2: CCAAATTGCCCAAGACGTCGGACTTGCACCTGTTTCATCCCGAACTCCATCCAAMNNATT CAGCATTGTTTG (SEQ ID NO: 4), the noncoding mutagenic oligonucleotide for position 117; 3: GAAACAGGTGCAAGTCCGAC GTCTTGGGCAA (SEQ ID NO: 5), a non-mutagenic coding strand primer with overlap to the mutagenic 117 primer; 4: GTTTTCTGTCCGCGGATCCATTCCTG (SEQ ID NO: 6), a noncoding strand primer flanking the unique SacII site of the open reading frame. A PCR fragment was generated by overlap extension of fragments a and b using primers 1 and 4. This fragment was restricted by XbaI and SacII and introduced into the equivalent sites into pLac3 containing the wild type castor Δ⁹-18:0-desaturase. The other five positions of castor Δ⁹-18:0-desaturase were mutated independently in an equivalent fashion to methods used for position 117.

Random point mutagenesis.

Random mutagenesis was performed on sequences encoding castor Δ⁹-18:0-ACP desaturase by single gene DNA shuffling (W. P. Stemmer, (1994)). The open reading frame was first amplified by PCR using the primers: GTGAGCGGATAACAATTTCACACAGTCTAGAAAT (SEQ ID NO: 7) which corresponds to the coding strand, and CACGAGGCCCTTTCGTCTTCAAGAATTCTC (SEQ ID NO: 8) which corresponds to the noncoding strand. Approximately 50-200 bp from the wild type castor open reading frame was digested with DNaseI to make fragments at random positions within the open reading frame. The gene was then assembled by primeness PCR, followed by amplification of the full open reading frame using 5′ and 3′ specific primers. This method incorporated primarily point mutations at high frequency. The mutagenesis method used here was arbitrarily chosen, any method of point mutagenesis could have been used to produce equivalent results.

Combinatorial full positional randomization.

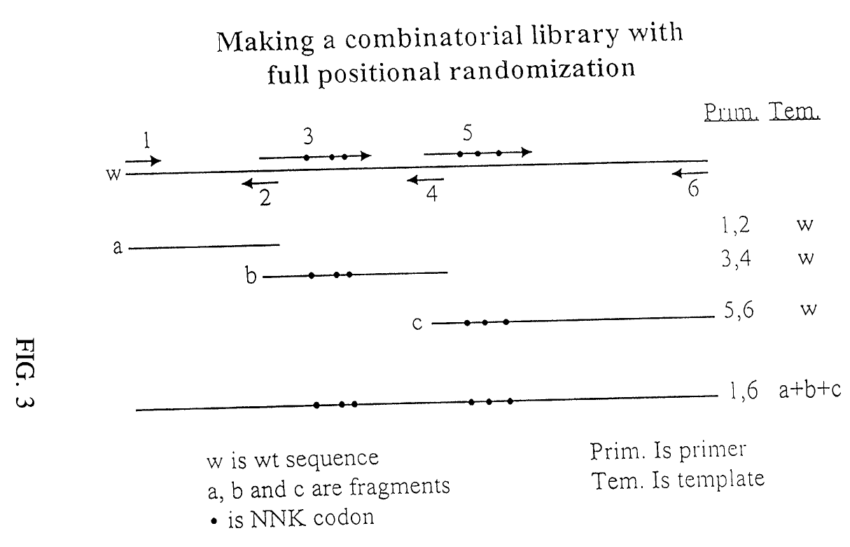

For the combinatorial 6 site-specific full positional randomization primers were engineered to contain NNK (where N refers to an equimolar mixture of G, A, T and C, and K refers to an equimolar mixture of G and T) for each of the six target codons. The full open reading frame was assembled and amplified by overlap extension PCR as shown in FIG. 3. Primers corresponding to the diagram were 1: GTGAGCGGATAA CAATTTCACACAGTCTAGAAAT (SEQ ID NO: 3), sequence flanking the unique XbaI site at the 5′ end of the open reading frame; 2: TTGATAAGTGGGAAGGGCTTCTTCCGTT (SEQ ID NO: 9), non-mutagenic noncoding primer; 3: AACGGAAGAAGCCCTTCCCACTTATCAAAC ANNKCTGAATNNKNNKGATGGAGTTCGGGATGAAAC (SEQ ID NO: 10), mutagenic coding strand primer; 4: TCCATTCCTGAACCAA TCAAATATTG (SEQ ID NO: 11), non-mutagenic noncoding strand primer; 5: TTGATTGGTTCAGGAATGGATNNKCGGNNKGAAAACAGTCCATACCT TNNKTTCATCTATACATCATTCC (SEQ ID NO: 12), mutagenic coding strand primer; 6: GCAAAAGCCAAAACGGTACCATCAGGATCA (SEQ ID NO: 13), noncoding non-mutagenic primer flanking the KpnI site. The three fragments were first amplified as shown in FIG. 3. They were isolated and amplified by overlap-extension PCR as described above for full positional randomization of T117. The final fragment was restricted using XbaI and KpnI, and introduced into the equivalent sites in pLac3 containing the wild type castor Δ⁹-18:0-ACP desaturase.

Selection of mutant desaturases with altered chain length specificity.

To facilitate determination of the function of a plant acyl-ACP desaturase in E. coli, an expression vector containing the gene for plant-type ferredoxin, the redox partner of the plant desaturase, was transformed into the MH13 E. coli and maintained under selective pressure. These cells, MH13(pACYC/LacAnFd) were used in the following experiments. MH13(pACYC/LacAnFd) were transformed with the resulting libraries of mutated 18:0-ACP desaturase under conditions appropriate for expression. To achieve this, clones were restricted with either XbaI and KpnI, or XbaI and EcoRI, and introduced into the corresponding sites of plasmid pLac3 containing the mature castor Δ⁹-desaturase open reading frame. The plasmid pLac contains the Lac promoter which can be induced using the chemical inducer ispropyl β-thiogalactopyranoside (IPTG). Selection media lacking unsaturated fatty acids was used to identify mutants with the ability to complement the unsaturated fatty acid auxotrophy. To confer survival under the selective conditions, a mutant desaturase would necessarily have an altered substrate chain length specificity of 16, 14 or fewer carbons. The selection for site directed mutants was performed in either liquid media or on agar plates. The selection for randomly generated mutants was performed on agar plates. Growth in liquid media involved several rounds of dilution and re-growth to enrich for mutations that resulted in the best complementation. DNA for all mutants identified in this fashion was isolated and reintroduced into the mutant E. coli cell line, which was subjected to another round of selection to confirm the phenotype. The DNA of the selected desaturases were sequenced and translated conceptually to identify the specific mutations incurred.

Enzyme analyses. For determination of biochemical parameters of the desaturase mutants, the open reading frame was excised by restriction with XbaI and KpnI and ligated into the corresponding sites of the plasmid pLac3, which put the mature castor Δ⁹-desaturase open reading frame under the control of the Lac promoter. The plasmid was expressed in the cell line BL21DE3 Gold (Novagen) for expression. Cells were grown to 0.5 OD600, induced by addition of 0.4 mM IPTG and harvested after four hours. The desaturase enzyme was extracted and purified to near homogeneity (90%) by HPLC cation exchange chromatography using Poros 20CM media (Perseptive Biosystems). Purified desaturase was assayed using C1-¹⁴C acyl-ACP of appropriate chain lengths. Substrate and products were converted to methyl esters and analyzed by argentation thin layer chromatography and phoshor-imaging. Specific activities with the different substrates were calculated (Cahoon et al., (1997)).

1. A mutant castor Δ⁹-18:0-ACP desaturase having one or more amino acid substitutions selected from the group consisting of:
a) Ala or Thr for Met at residue 114 of SEQ ID NO: 1;
b) Arg for Thr at residue 117 of SEQ ID NO: 1;
c) Gly or Ala for Leu at residue 118 of SEQ ID NO: 1;
d) Val or Leu for Pro at residue 179 of SEQ ID NO: 1;
e) Val or Ser for Thr at residue 181 of SEQ ID NO: 1; and
f) Leu for Gly at residue 188 of SEQ ID NO: 1.

2. The mutant castor Δ⁹-18:0-ACP desaturase of claim 1 which has the amino acid substitution Arg for Thr at residue 117 of SEQ ID NO: 1.

3. The mutant castor Δ⁹-18:0-ACP desaturase of claim 1 which has the amino acid substitution Arg for Thr at residue 117 of SEQ ID NO: 1 and Leu for Gly at residue 188 of SEQ ID NO: 1.

4. The mutant castor Δ⁹-18:0-ACP desaturase of claim 1 which contains each of the following amino acid substitutions:
a) Ala for Met at residue 114 of SEQ ID NO: 1;
b) Arg for Thr at residue 117 of SEQ ID NO: 1;
c) Gly for Leu at residue 118 of SEQ ID NO: 1;
d) Val for Pro at residue 179 of SEQ ID NO: 1;
e) Val for Thr at residue 181 of SEQ ID NO: 1; and
f) Leu for Gly at residue 188 of SEQ ID NO: 1.

5. The mutant castor Δ⁹-18:0-ACP desaturase of claim 1 which contains each of the following amino acid substitutions:
a) Thr for Met at residue 114 of SEQ ID NO: 1;
b) Arg for Thr at residue 117 of SEQ ID NO: 1;
c) Ala for Leu at residue 118 of SEQ ID NO: 1;
d) Leu for Pro at residue 179 of SEQ ID NO: 1;
e) Ser for Thr at residue 181 of SEQ ID NO: 1; and
f) Leu for Gly at residue 188 of SEQ ID NO: 1.

6. A DNA expression construct comprising, in expressible form, a nucleic acid sequence which encodes a mutant castor Δ⁹-18:0-ACP desaturase having one or more amino acid substitutions selected from the group consisting of:
a) Ala or Thr for Met at residue 114 of SEQ ID NO: 1;
b) Arg for Thr at residue 117 of SEQ ID NO: 1;
c) Gly or Ala for Leu at residue 118 of SEQ ID NO: 1;
d) Val or Leu for Pro at residue 179 of SEQ ID NO: 1;
e) Val or Ser for Thr at residue 181 of SEQ ID NO: 1; and
f) Leu for Gly at residue 188 of SEQ ID NO: 1.

7. A DNA expression construct comprising, in expressible form, a nucleic acid sequence which encodes a mutant castor Δ⁹-18:0-ACP desaturase having each of the following amino acid substitutions:
a) Ala for Met at residue 114 of SEQ ID NO: 1;
b) Arg for Thr at residue 117 of SEQ ID NO: 1;
c) Gly for Leu at residue 118 of SEQ ID NO: 1;
d) Val for Pro at residue 179 of SEQ ID NO: 1;
e) Val for Thr at residue 181 of SEQ ID NO: 1; and
f) Leu for Gly at residue 188 of SEQ ID NO: 1.

8. A DNA expression construct comprising, in expressible form, a nucleic acid sequence which encodes a mutant castor Δ⁹-18:0-ACP desaturase having each of the following amino acid substitutions:
a) Thr for Met at residue 114 of SEQ ID NO: 1;
b) Arg for Thr at residue 117 of SEQ ID NO: 1;
c) Ala for Leu at residue 118 of SEQ ID NO: 1;
d) Leu for Pro at residue 179 of SEQ ID NO: 1;
e) Ser for Thr at residue 181 of SEQ ID NO: 1; and
f) Leu for Gly at residue 188 of SEQ ID NO: 1.

9. A cell transformed with the DNA expression construct comprising, in expressible form, a nucleic acid sequence which encodes a mutant castor Δ⁹-18:0-ACP desaturase having one or more amino acid substitutions selected from the group consisting of:
a) Ala or Thr for Met at residue 114 of SEQ ID NO: 1;
b) Arg for Thr at residue 117 of SEQ ID NO: 1;
c) Gly or Ala for Leu at residue 118 of SEQ ID NO: 1;
d) Val or Leu for Pro at residue 179 of SEQ ID NO: 1;
e) Val or Ser for Thr at residue 181 of SEQ ID NO: 1; and
f) Leu for Gly at residue 188 of SEQ ID NO: 1.

10. The cell of claim 9 which is a prokaryotic cell.

11. The cell of claim 9 which is an eukaryotic cell.

12. The cell of claim 11 which is a plant cell.

13. A transgenic plant expressing a nucleic acid sequence which encodes a mutant castor Δ⁹-18:0-ACP desaturase having one or more amino acid substitutions selected from the group consisting of:
a) Ala or Thr for Met at residue 114 of SEQ ID NO: 1;
b) Arg for Thr at residue 117 of SEQ ID NO: 1;
c) Gly or Ala for Leu at residue 118 of SEQ ID NO: 1;
d) Val or Leu for Pro at residue 179 of SEQ ID NO: 1;
e) Val or Ser for Thr at residue 181 of SEQ ID NO: 1; and
f) Leu for Gly at residue 188 of SEQ ID NO: 1.

14. The transgenic plant of claim 13 which is Arabidopsis thaliana.

15. A transgenic plant expressing a nucleic acid sequence which encodes a mutant castor Δ⁹-18:0-ACP desaturase having each of the following amino acid substitutions:
a) Ala for Met at residue 114 of SEQ ID NO: 1;
b) Arg for Thr at residue 117 of SEQ ID NO: 1;
c) Gly for Leu at residue 118 of SEQ ID NO: 1;
d) Val for Pro at residue 179 of SEQ ID NO: 1;
e) Val for Thr at residue 181 of SEQ ID NO: 1; and
f) Leu for Gly at residue 188 of SEQ ID NO: 1.

16. The transgenic plant of claim 15 which is Arabidopsis thaliana.

17. A transgenic plant expressing a nucleic acid sequence which encodes a mutant castor Δ⁹-18:0-ACP desaturase having each of the following amino acid substitutions:
a) Thr for Met at residue 114 of SEQ ID NO: 1;
b) Arg for Thr at residue 117 of SEQ ID NO: 1;
c) Ala for Leu at residue 118 of SEQ ID NO: 1;
d) Leu for Pro at residue 179 of SEQ ID NO: 1;
e) Ser for Thr at residue 181 of SEQ ID NO: 1; and
f) Leu for Gly at residue 188 of SEQ ID NO: 1.

18. The transgenic plant of claim 17 which is Arabidopsis thaliana.

19. A DNA expression construct comprising, in expressible form, a nucleic acid sequence which encodes a mutant castor Δ⁹-18:0-ACP desaturase which has the amino acid substitution Arg for Thr at residue 117 of SEQ ID NO: 1.

20. A cell transformed with a DNA expression construct comprising, in expressible form, a nucleic acid sequence which encodes a mutant castor Δ⁹-18:0-ACP desaturase which has the amino acid substitution Arg for Thr at residue 117.

21. The cell of claim 20 which is prokaryotic.

22. The cell of claim 20 which is eukaryotic.

23. The cell of claim 22 which is a plant cell.

24. A transgenic plant expressing a nucleic acid sequence which encodes a mutant castor Δ⁹-18:0-ACP desaturase which has the amino acid substitution Arg for Thr at residue 117.

25. The transgenic plant of claim 24 which is Arabidopsis thaliana.

26. A cell transformed with a DNA expression construct comprising, in expressible form, a nucleic acid sequence which encodes a mutant castor Δ⁹-18:0-ACP desaturase which contains each of the following amino acid substitutions:
a) Ala for Met at residue 114;
b) Arg for Thr at residue 117;
c) Gly for Leu at residue 118;
d) Val for Pro at residue 179;
e) Val for Thr at residue 181; and
f) Leu for Gly at residue 188.

27. The cell of claim 26 which is prokaryotic.

28. The cell of claim 26 which is eukaryotic.

29. The cell of claim 28 which is a plant cell.

30. A mutant castor Δ⁹-18:0-ACP desaturase which has an amino acid substitution of Phe for Thr at residue 181 of SEQ ID NO: 1.

31. A mutant castor Δ⁹-18:0-ACP desaturase which has the amino acid substitution Trp for Thr at residue 181 of SEQ ID NO: 1.

32. A DNA expression construct comprising, in expressible form, a nucleic acid sequence which encodes a mutant castor Δ⁹-18:0-ACP desaturase which has an amino acid substitution of Phe for Thr at residue 181 of SEQ ID NO: 1.

33. A DNA expression construct comprising, in expressible form, a nucleic acid sequence which encodes a mutant castor Δ⁹-18:0-ACP desaturase which has an amino acid substitution of Trp for Thr at residue 181 of SEQ ID NO: 1.

34. A cell transformed with a DNA expression construct comprising, in expressible form, a nucleic acid sequence which encodes a mutant castor Δ⁹-18:0-ACP desaturase which has an amino acid substitution of Phe for Thr at residue 181 of SEQ ID NO: 1.

35. The cell of claim 34 which is a prokaryotic cell.

36. The cell of claim 34 which is a eukaryotic cell.

37. The cell of claim 36 which is a plant cell.

38. A transgenic plant expressing a nucleic acid sequence which encodes a mutant castor Δ⁹-18:0-ACP desaturase which has an amino acid substitution of Phe for Thr at residue 181 of SEQ ID NO: 1.

39. The transgenic plant of claim 38 which is Arabidopsis thaliana.

40. A method for specifically altering a function of a protein through directed mutagenesis, comprising:
a) identifying candidate amino acid positions of the protein which when mutated are predicted to alter the function;
b) generating a library of mutants of the protein, the mutants being generated by randomization of the amino acid encoded at each candidate position, in combination with randomization of every other candidate position; and
c) identifying mutants which exhibit the desired specific alteration of function from the library of mutants.

41. The method of claim 40 wherein the candidate amino acid positions are identified by a combination of methods.

42. The method of claim 40 wherein the candidate positions comprise positions of amino acids which directly participate in the function which is to be altered.

43. The method of claim 42 wherein the candidate positions further comprise positions of amino acids which indirectly participate in the function which is to be altered.

44. The method of claim 40 wherein the candidate positions are identified by random mutagenesis.

45. The method of claim 40 wherein the candidate positions are identified by structural analysis of the protein.

46. The method of claim 40 wherein the candidate positions are identified by sequence analysis and comparison to related proteins.

47. The method of claim 40 wherein the library of mutants is generated by overlap extension PCR.

48. The method of claim 40 wherein mutants which exhibit the desired alteration of function are identified by a selective screening process.

49. The method of claim 40 wherein the protein is an enzyme.

50. The method of claim 49 wherein the enzyme is castor Δ⁹-18:0-ACP desaturase.

51. The method of claim 49 wherein substrate specificity of the enzyme is altered.

52. The method of claim 49 wherein in vivo activity of the enzyme is altered.

53. The method of claim 49 wherein in vitro activity of the enzyme is altered.

54. The method of claim 49 wherein in vivo and in vitro activity of the enzyme is altered.

55. The method of claim 40 wherein the protein is a ligand binding protein.

56. The method of claim 55 wherein the in vivo ligand binding specificity of the protein is altered.

57. The method of claim 55 wherein the in vitro ligand binding specificity of the protein is altered.

58. The method of claim 55 wherein the in vivo and in vitro ligand binding specificity of the protein are altered.

59. The method of claim 40 wherein the protein is a structural protein.
